(19) United States
(12) Patent Application Publication
(10) Pub. No.: US 2008/0292645 A1
(43) Pub. Date: Nov. 27, 2008

(54) Antibody or Antibody Fragment Coupled with an Immunogenic Agent

(76) Inventors: Anthony Courtois, Saint Renan (FR); Christian Berthou, Brest (FR); Anne Loussouarn, Le Relecq Kerhuon (FR); Jean Guezennec, Plouzane (FR); Claire Boisset, Roscoff (FR); Stephanie Gac-Breton, Plouescat (FR)

Correspondence Address: YOUNG & THOMPSON, 209 Madison Street, Suite 500, ALEXANDRIA, VA, 22314, US

(21) Appl. No.: 11/915,341

(22) PCT Filed: May 24, 2006
(86) PCT No.: PCT/FR2006/001196

§ 371 Date: May 12, 2008

(30) Foreign Application Priority Data
May 24, 2005 (FR) 0505219

(51) Int. Cl. A61K 39/395; C07K 16/18; A61P 31/00; C07K 1/00

(52) U.S. Cl. 424/178.1; 530/391.1; 530/402

(57) ABSTRACT

An immunogenic conjugate includes a target cell-specific circulating molecule and at least one immunogenic agent, the immunogenic agent being coupled by any appropriate means with the circulating molecule. Also described is a method for preparing the immunogenic conjugate and its use for treating cancers or autoimmune diseases.

The present invention relates to the immunology field, and the subject thereof is a novel conjugate capable of inducing at least an immune response in a mammal. Such a compound is also referred to as immunogenic conjugate. The present invention also relates to a method for obtaining such an immunogenic conjugate. Finally, it relates to the use of this novel immunogenic conjugate in a pharmaceutical composition, in particular for the treatment of cancers or any diseases treated with antibodies.

There currently exist about fifteen therapeutic monoclonal antibodies used in pathologies as diverse as cancers, graft rejection, infectious diseases, or the treatment of inflammatory or allergic pathologies.

Rituximab was the first monoclonal antibody approved by the FDA (Foods and Drugs Administration) in the treatment of B lymphomas (IDEC pharmaceuticals). This chimeric antibody is directed against a target molecule very specific for B lymphocytes, the CD20 antigen, which is expressed in large amounts at the surface of lymphocytes during lymphopoiesis from the pre-B stage to the mature B lymphocyte stage, but is absent from hematopoietic stem cells and plasmocytes. This antibody is made up of the variable regions of an anti-CD20 murine antibody fused with the constant regions of human kapa light chains and gamma 1 heavy chains (Reff et al., 1994, Depletion of B cells in vivo by a chimeric mouse human monoclonal antibody to CD20. Blood 83(2): 435-45). The fact that this antibody is effective and well tolerated makes it a therapeutic tool of choice in the treatment of B-type lymphomas and autoimmune diseases.

Therapeutic monoclonal antibodies, in particular rituximab, induce a cytotoxic activity due to the fact that they bind to the surface of target tumor cells. This cytotoxic activity is reflected by activation of the complement system (CDC and/or CDCC) and/or the induction of an antibody-dependent cytotoxicity (ADCC), inducing, as a result, apoptosis of the target tumor cells.

In order to increase their cytotoxic activity in the context of antitumor therapies, the monoclonal antibodies can be coupled to active agents, for example radioisotopes, or to toxins. While the noncoupled monoclonal antibodies induce a cytotoxic response merely due to the fact that they bind to the surface of the target cells, the coupled antibodies also have a role as a vector for active agents.

The radiolabeled antibodies have a cytolytic effect on tumor cells essentially due to the radioactive emission of the radioisotope. Their mechanism of action lies, firstly, in the specific recognition of the tumor cells targeted by the antibody and, secondly, in the irradiation of the tumor cells by the radioisotope carried by the antibody.

The immunotoxins correspond to antibodies coupled to toxins or to subunits of said toxins. The toxins are nonspecific and extremely powerful, lethal for cells at very low concentrations. After binding to the surface of the target cell, the antibody-toxin complex is internalized into the cells. The immunotoxins destroy the target cells in particular by inhibiting protein synthesis essential to the survival of the cells.

While clinical studies have shown positive results, the development of antitumor treatments with radioisotopes or immunotoxins is confronted by two major problems: a low efficacy of certain radioisotopes or immunotoxins and a toxicity which is nonspecific and difficult to control. The low efficacy of the monoclonal antibodies coupled to radioisotopes or toxins could be due to the fact that their mechanism of action is essentially independent of the immune system. In fact, the destruction of the target cells is not mainly induced by the recruitment of the immune system, but by the irradiation of the cells, by the inhibition of protein synthesis and by an abscopal effect.

To date, antibodies or antibody fragments and also other types of vectors (¹⁸⁶Re-bisphosphonates, ⁶⁸Ga-DOTA-albumin, HPMA-toxins, etc.) have been used as circulating molecules for carrying radioisotopes or toxins to target tumor cells.

One objective of the invention is to couple, for therapeutic purposes, at least one immunogenic agent to a target cell-specific circulating molecule, with the aim of creating an immunogenic conjugate capable of inducing or amplifying a specific immune response against these target cells. According to the invention, the immunogenic agent can be coupled by any appropriate means to a circulating agent having at least one site for specific binding to the target cell. A subject of the invention is therefore an immunogenic conjugate comprising a target cell-specific circulating molecule and at least one immunogenic agent, said immunogenic agent being coupled by any appropriate means to the circulating molecule.

In particular, one objective of the present invention is to amplify the specific cytotoxic activity of monoclonal antibodies or of antibody fragments by coupling them to immunogenic agents capable of inducing at least an immune response in a mammal. The amplification of the cytotoxic activity of the antibody is in particular reflected by an increase in the activation of the complement system, but also a stimulation of the various cellular effectors of the immune system (ADCC) (Watier et al., 1996, Human NK cell mediated direct and IgG dependent cytotoxicity against xenogeneic porcine endothelial cells, Transpl. Immunol., 4(4): 293-9).

The term “circulating molecule” is intended to mean any molecule capable of circulating in the blood and/or lymphatic system, and/or of diffusing across the blood-brain barrier and/or the organs; said circulating molecule preferably being an immunoglobulin or a fragment thereof.

The term “target cell” is intended to mean any cell involved in autoimmune and/or cancerous diseases.

The term “target cell-specific molecule” is intended to mean any molecule capable of specifically recognizing a motif, in particular an antigen, receptor, or a protein, expressed at the surface of the target cell.

The term “immunogenic agent” is intended to mean any molecule capable of inducing a cytotoxic activity reflected by an activation of the complement system and/or the induction of an antibody-dependent cytotoxicity.

The term “to couple” is intended to mean the creation of a bond between a circulating molecule and at least one immunogenic agent, said bond being in particular a covalent bond between the circulating molecule and the immunogenic agent or a bond by means of a linker between the circulating molecule and the immunogenic agent.

The expression “activation of the complement system” is intended to mean the activation of the immune response via the conventional pathway of the complement system comprising a cascade of enzymatic reactions involving about twenty proteins, or via the alternate pathway of the complement system or the lectin pathway. These proteins are activated in a serial proteolytic chain which generates lysis by osmotic shock of the target cell (CDC), thus inducing the production of peptides with biological activities (CDCC).

The expression “induction of an antibody-dependent cytotoxicity” is intended to mean the induction of an immune response in which killer cells carrying antibody Fc fragment receptors, in particular natural killer cells (NK), and macrophages, recognize the target cells coated with specific antibodies.

In a first embodiment of the invention, the immunogenic conjugate is characterized in that the coupling between the immunogenic agent and the circulating molecule is direct, i.e. said at least one immunogenic agent is coupled to the circulating molecule by a covalent bond.

In a second embodiment of the invention, the immunogenic conjugate is characterized in that said at least one immunogenic agent is coupled to the circulating molecule by means of a linker.

The term “linker” is intended to mean any reactive molecule having at least one reactive function capable of forming a bond with a reactive function of the circulating molecule and at least one reactive function capable of forming a bond with at least one reactive function of the immunogenic agent. The term “reactive function” is intended to mean any functional group capable of bonding by covalent bonding with another functional group, for example, in particular, amino, thiol, hydroxyl, carbonyl, activated ester, hydrazine, isocyanate, halogen, groups, etc.

The linker is preferably heterobifunctional, i.e. carrying distinct reactive functions in order to prevent unwanted bonds being formed between two immunogenic agents or between two circulating molecules. The linker is preferably chosen from the following commercially available reagents:
N-acetylhomocysteine thiolactone, S-acetyl-mercaptosuccinic anhydride, 3-mercaptopropion-imidate, 4-mercaptobutyramide or its cyclic form the Traut's reagent (2-iminothiolane), SMPT (4-succinimidyloxycarbonylmethyl-α-[2-pyridyldithio]-toluene), SPDP (N-succinimidyl 3-(2-pyridyl-dithio)propionate), LC-SPDP (succinimidyl 6-(3-[2-pyridyldithio]propionamido)hexanoate), sulfo-LC-SPDP, PDP hydrazide (3-(2-pyridyldithio)propionyl-hydrazide, DTT (dithiothreitol), TCEP (tris(2-carboxyethyl)phosphine hydrochloride), SATA (N-succinimidyl-S-acetylthioacetate), SATP (N-succinimidyl-S-acetylthiopropionate), 3-(3-acetyl-thiopropionyl)thiazolidine-2-thione, 3-(3-p-meth-oxybenzylthiopropionyl)thiazolidine-2-thione for the introduction of thiol capable of reacting with a maleimide function or a halogenated derivative present on the circulating molecule;
SMCC (succinimidyl 4-(N-maleimidomethyl)cyclo-hexane-1-carboxylate), sulfo-SMCC, LC-SMCC (succinimidyl 4-(N-maleimidomethyl)cyclohexane-1-carboxy-(6-amidocaproate)), KMUH (N-(κ-maleimido-undecanoic acid)hydrazide), KMUS (N-(κ-maleimido-undecanoyloxy)succinimide ester), sulfo-KMUS, PMPI (N-(p-maleimidophenyl)isocyanate), SMPB (succinimidyl 4-(p-maleimidophenyl)butyrate), sulfo-SMPB, SMPH (succinimidyl-6-(β-maleimidopropionamido)-hexanoate), AMAS (N-(α-maleimidoacetoxy)succinimide ester), BMPH (N-(β-maleimidopropionic acid)hydrazide), BMPS (N-(β-maleimidopropyloxy)-succinimide ester), MBS (m-maleimidobenzoyl-N-hydroxysuccinimide ester) and sulfo-MBS, EMCS (N-(ε-maleimidocaproyloxy)succinimide ester) and sulfo-EMCS, SMPB (succinimidyl 4-(P-maleimido-phenyl)butyrate), sulfo-SMPB, EMCH ((N-ε-maleimidocaproic acid)hydrazide) for the introduction of maleimide capable of reacting with a thiol function present on the circulating molecule;
SANH (succinimidyl 4-hydrazinonicotinate acetone hydrazone) and C6-SANH, succinimidyl 4-hydrazido-terephthalate hydrochloride, KMUH (N-(κ-maleimido-undecanoic acid)hydrazide), BMPH (N-β-maleimido-propionic acid)hydrazide, PDP hydrazide (3-(2-pyridyldithio)propionyl hydrazide), EMCH for the introduction of hydrazine/hydrazide capable of reacting with an aldehyde function present on the circulating molecule;
SFB (succinimidyl 4-formylbenzoate) and C6-SFB, sodium meta-periodate for the introduction of aldehyde capable of reacting with a hydrazine/hydrazide function present on the circulating molecule;
PMPI (N-[p-maleimidophenyl]isocyanate) for the introduction of an isocyanate function capable of reacting with an alcohol function present on the circulating molecule;
SBAP (succinimidyl 3-[bromoacetamido]propionate), SIA (N-succinimidyl iodoacetate), SIAB (N-succinimidyl[4-iodoacetyl]aminobenzoate), sulfo-SIAB for the introduction of halogenated derivatives capable of reacting with a thiol function present on the circulating molecule.

According to a preferred embodiment of the invention, the linker is sulfo-SMCC.

In a preferred embodiment of the invention, said circulating molecule is a mono-specific or multi-specific monoclonal antibody, or an antibody fragment, preferably an antibody fragment having one or more recognition sites and sites for binding to one or more target molecules, more preferably a Fab fragment.

Antibodies or immunoglobulins (Ig) are composed of 2 identical heavy chains (H chains) and 2 light chains (L chains) forming a Y-shaped structure stabilized by disulfide bonds. The ends of the arms of the Y contain variable regions of the heavy and light chains which bind to an antigen in the case of a monospecific monoclonal antibody and to distinct antigens in the case of multispecific monoclonal antibodies. The regions are termed variable due to the presence of three hypervariable zones or complementarity-determining regions (CDRs). It is this great variability which explains the ability of antibodies to recognize various antigens.

The base of the Y corresponds to the constant region (F_{c}) and is not involved in recognition and specific antigen binding. This constant region has effector functions, and is in particular involved in the binding of the antibody to cell receptors and complement binding. Immunoglobulins (Igs) are divided up into classes and subclasses according to the structure of this Fc region. 5 immunoglobulin classes exist, IgG, IgA, IgM, IgE and IgD, some being themselves divided up into IgG subclasses: IgG1, IgG2, IgG3 and IgG4. Thus, IgG1, IgG2, IgG3 and IgM immunoglobulins are capable of activating the complement system, while IgG1 and IgG3 immunoglobulins appear to activate ADCC more effectively than IgG2 and IgG4 immunoglobulins. The study of the fragments obtained after proteolytic enzyme digestion on immunoglobulins has made it possible to elucidate the relationships that exist between the structure and the function of antibodies. The antibody fragment used as circulating molecule in the invention is generally obtained after the action of proteolytic enzymes, preferably pepsin or papain. Pepsin generally cleaves the antibody molecule below the disulfide bridge(s) which link(s) the two heavy chains. After the action of pepsin, the (Fab′)₂ fragment obtained corresponds to the two Fab fragments linked by one or more disulfide bridges, and Fc fragment degradation products. On the other hand, under the action of papain, the antibody molecule is generally cleaved into three fragments corresponding to two Fab fragments and one Fc fragment.

Each Fab (ab for antigen binding) fragment is composed of a light chain and of a fragment of the heavy chain. The essential property of a Fab fragment is to bind to an antigen and to induce apoptosis. The Fc (c for crystallizable) fragment corresponding to the constant region therefore carries the effector capacities of the antibody, is in particular capable of binding the C1q component of complement (CDC) and the Fc receptors of effector cells.

Thus, antibodies are capable not only of specifically recognizing a target molecule, in particular an antigen, and of binding thereto via their variable region (Fab), but also of activating the complement system and recruiting immunocompetent cells via their constant region (Fc). Antibodies are characterized by their molecular plasticity which makes them capable of recognizing antigens and of recruiting effector cells.

Multispecific antibodies are antibodies, which are often monoclonal, in which the antigen-binding sites are specific for distinct antigenic determinants. They are produced by a chemical attachment, the fusion of hybridoma cells, or by molecular genetics techniques. They function as principal mediators of targeted cell cytotoxicity and have been found to be effective in the targeting of drugs, toxins, radiolabeled haptens, and effector cells against diseased, mainly tumoral, tissues.

In a preferred embodiment of the invention, the immunogenic conjugate comprises an immunogenic agent coupled to a Fab fragment. The Fab fragment comprises advantageous pharmacokinetic properties such as the rapidity of action and of diffusion of the conjugate, whereas the immunogenic agent confers on the conjugate the ability to induce strong immune responses, in particular by activating the complement system.

In a preferred embodiment of the invention, said immunogenic agent comprises at least one oligosaccharide, which itself comprises at least one motif capable of being recognized by at least one antibody and/or one soluble protein of human complement and/or by a receptor expressed at the surface of immune system effector cells.

By way of examples of oligosaccharides suitable for the purposes of the invention, mention may in particular be made of disaccharides or trisaccharides, preferably comprising the GalαGal epitope, more preferably in particular Gal-(α.1,3)-Gal, Gal-(α.1,3)-Gal-R where R may be a monosaccharide, an amine function, a hydroxyl function, a carboxylate function, or an organic or inorganic substituent. The GalαGal epitope has been identified as being responsible for hyperacute xenograft rejection which is observed from the first minutes of transplantation onward; the rejection is due to the recognition, by circulating natural human antibodies of IgG and IgM type, of the epitope present at the surface of the cells of the transplanted organ. This epitope is therefore particularly toxic for cells which express it since its recognition by the natural human antibodies activates the complement system and the recruitment of effector cells, resulting in cell lysis. In the context of the invention, the oside structures of blood groups (for example: A, B, Lewis, etc.) can be used as immunogenic agents, but also agents that are active in the chemotaxis of immune system effector cells, such as platelet activating factor analogs.

Unlike treatments with monoclonal antibodies based on the administration of immune molecules, the principle of the vectorization of molecules that are active on the complement system would make it possible to trigger an immune reaction in the close environment of the target cells and to initiate a localized inflammatory reaction, which should make it possible to overcome the mechanisms of resistance that these tumors have been able to set up with respect to the complement system. This local activation of the immune system may be due to a high density of immunogenic agents at the surface of the target cells, which would not lead to any activation of the immune system in the blood stream.

Another objective of the invention is to propose a method for preparing the immunogenic conjugate as described above, comprising:
optionally, the creation of reactive functions in the circulating molecule and in the immunogenic agent,
a coupling reaction between the circulating molecule and at least one immunogenic agent.

In one embodiment of said method, in which the circulating molecule and the immunogenic agent do not comprise reactive functions, reactive functions are optionally artificially created in the circulating molecule and in the immunogenic agent before the coupling reaction.

The term “creating” is intended to mean introducing or converting a functional group naturally present into a reactive function.

According to a preferred embodiment of the invention, the modification of the circulating molecule is preferably carried out by addition of a reagent which makes it possible to artificially create at least one reactive function on the circulating molecule. The reagent is preferably chosen from commercially available reagents, in particular:
N-acetylhomocysteine thiolactone, S-acetyl-mercaptosuccinic anhydride, 3-mercaptopropion-imidate, 4-mercaptobutyramide or its cyclic form the Traut's reagent (2-iminothiolane), SMPT (4-succinimidyloxycarbonylmethyl-α-[2-pyridyldithio]-toluene), SPDP (N-succinimidyl 3-(2-pyridyl-dithio)propionate), LC-SPDP (succinimidyl 6-(3-[2-pyridyldithio]propionamido)hexanoate), sulfo-LC-SPDP, PDP hydrazide (3-(2-pyridyldithio)propionyl-hydrazide, DTT (dithiothreitol), TCEP (tris(2-carboxyethyl)phosphine hydrochloride), SATA (N-succinimidyl-S-acetylthioacetate), SATP(N-succinimidyl-S-acetylthiopropionate), 3-(3-acetyl-thiopropionyl)thiazolidine-2-thione, 3-(3-p-meth-oxybenzylthiopropionyl)thiazolidine-2-thione for the introduction of thiol;
SMCC (succinimidyl 4-(N-maleimidomethyl)cyclo-hexane-1-carboxylate), sulfo-SMCC, LC-SMCC (succinimidyl 4-(N-maleimidomethyl)cyclohexane-1-carboxy-(6-amidocaproate)), KMUH(N-(κ-maleimido-undecanoic acid)hydrazide), KMUS (N-(κ-maleimido-undecanoyloxy)succinimide ester), sulfo-KMUS, PMPI (N-(p-maleimidophenyl)isocyanate), SMPB (succinimidyl 4-(p-maleimidophenyl)butyrate), sulfo-SMPB, SMPH (succinimidyl-6-(β-maleimidopropionamido)-hexanoate), AMAS (N-(α-maleimidoacetoxy)succinimide ester), BMPH(N-(β-maleimidopropionic acid)hydrazide), BMPS(N-(β-maleimidopropyloxy)-succinimide ester), MBS (m-maleimidobenzoyl-N-hydroxysuccinimide ester) and sulfo-MBS, EMCS (N-(ε-maleimidocaproyloxy)succinimide ester) and sulfo-EMCS, SMPB (succinimidyl 4-(P-maleimido-phenyl)butyrate), sulfo-SMPB, EMCH ((N-ε-male-imidocaproic acid)hydrazide) for the introduction of maleimide;
SANH (succinimidyl 4-hydrazinonicotinate acetone hydrazone) and C6-SANH, succinimidyl 4-hydrazido-terephthalate hydrochloride for the introduction of hydrazine/hydrazide;
SFB (succinimidyl 4-formylbenzoate) and C6-SFB, sodium meta-periodate for the introduction of aldehyde;

SBAP (succinimidyl 3-[bromoacetamido]propionate), SIA (N-succinimidyl iodoacetate), SIAB (N-succinimidyl[4-iodoacetyl]aminobenzoate), sulfo-SIAB for the introduction of halogenated derivatives that are reactive with respect to thiols.

SATA (N-succinimidyl-S-acetylthioacetate) is particularly preferred. 2-Iminothiolane (Traut's reagent) is also particularly preferred.

When the modification reaction is complete, the modified circulating molecule is preferably dialyzed, or ultrafiltered, more preferably at a cutoff threshold of less than 25 000 Da in the case of a Fab fragment, and at a cutoff threshold of less than 75 000 Da in the case of the whole antibody.

According to a preferred embodiment of the invention, the immunogenic agent may be modified in order to reveal a reactive function on the agent: a deacetylation step is carried out, preferably by addition of a solution of sodium hydroxide and sodium borohydride. The modified immunogenic agent may be purified on a steric exclusion column and/or ion exchange column and/or by HPLC, and then may optionally be lyophilized.

The reactive functions naturally present or artificially created in the circulating molecule and the immunogenic agent may or may not be complementary.

The term “complementary” is intended to mean reactive functions which are capable of creating covalent bonds with one another.

In a first embodiment of the method of the invention, in which the reactive functions naturally present or artificially created in the circulating molecule and the immunogenic agent are complementary, the coupling is carried out by reacting the circulating molecule with the immunogenic agent under conditions which allow the creation of covalent bonds between the complementary reactive functions.

In a second embodiment of the method of the invention, in which the reactive functions naturally present or artificially created in the circulating molecule and the immunogenic agent are not complementary, the coupling is carried out in the presence of a linker as defined above. Preferably, this linker is heterobifunctional and is chosen from the following commercially available reagents:
N-acetylhomocysteine thiolactone, S-acetyl-mercaptosuccinic anhydride, 3-mercaptopropion-imidate, 4-mercaptobutyramide or its cyclic form the Traut's reagent (2-iminothiolane), SMPT (4-succinimidyloxycarbonylmethyl-α-[2-pyridyldithio]-toluene), SPDP(N-succinimidyl 3-(2-pyridyl-dithio)propionate), LC-SPDP (succinimidyl 6-(3-[2-pyridyldithio]propionamido) hexanoate), sulfo-LC-SPDP, PDP hydrazide (3-(2-pyridyldithio)propionyl-hydrazide, DTT (dithiothreitol), TCEP (tris(2-carboxyethyl)phosphine hydrochloride), SATA (N-succinimidyl-S-acetylthioacetate), SATP (N-succinimidyl-S-acetylthiopropionate), 3-(3-acetyl-thiopropionyl)thiazolidine-2-thione, 3-(3-p-meth-oxybenzylthiopropionyl) thiazolidine-2-thione for the introduction of thiol capable of reacting with a maleimide function or a halogenated derivative present on the circulating molecule;
SMCC (succinimidyl 4-(N-maleimidomethyl)cyclo-hexane-1-carboxylate), sulfo-SMCC, LC-SMCC (succinimidyl 4-(N-maleimidomethyl)cyclohexane-1-carboxy-(6-amidocaproate)), KMUH (N-(κ-maleimido-undecanoic acid)hydrazide), KMUS (N-(κ-maleimido-undecanoyloxy) succinimide ester), sulfo-KMUS, PMPI (N-(p-maleimidophenyl) isocyanate), SMPB (succinimidyl 4-(p-maleimidophenyl) butyrate), sulfo-SMPB, SMPH (succinimidyl-6-(β-maleimidopropionamido)-hexanoate), AMAS (N-(α-maleimidoacetoxy)succinimide ester), BMPH (N-(β-maleimidopropionic acid) hydrazide), BMPS (N-(β-maleimidopropyloxy)-succinimide ester), MBS (m-maleimidobenzoyl-N-hydroxysuccinimide ester) and sulfo-MBS, EMCS (N-(ε-maleimidocaproyloxy) succinimide ester) and sulfo-EMCS, SMPB (succinimidyl 4-(P-maleimido-phenyl)butyrate), sulfo-SMPB, EMCH ((N-ε-maleimidocaproic acid)hydrazide) for the introduction of maleimide capable of reacting with a thiol function present on the circulating molecule;
SANH (succinimidyl 4-hydrazinonicotinate acetone hydrazone) and C6-SANH, succinimidyl 4-hydrazido-terephthalate hydrochloride, KMUH (N-(κ-maleimido-undecanoic acid)hydrazide), BMPH (N-β-maleimido-propionic acid)hydrazide, PDP hydrazide (3-(2-pyridyldithio)propionyl hydrazide), EMCH for the introduction of hydrazine/hydrazide capable of reacting with an aldehyde function present on the circulating molecule;
SFB (succinimidyl 4-formylbenzoate) and C6-SFB, sodium meta-periodate for the introduction of aldehyde capable of reacting with a hydrazine/hydrazide function present on the circulating molecule;
PMPI (N-[p-maleimidophenyl]isocyanate) for the introduction of an isocyanate function capable of reacting with an alcohol function present on the circulating molecule;
SBAP (succinimidyl 3-[bromoacetamido]propionate), SIA (N-succinimidyl iodoacetate), SIAB (N-succinimidyl[4-iodoacetyl]aminobenzoate), sulfo-SIAB for the introduction of halogenated derivatives capable of reacting with a thiol function present on the circulating molecule.

In a preferred embodiment of the method of the invention, the coupling of the circulating molecule and of the immunogenic agent is carried out in several successive steps so that unwanted bonds do not form between the circulating molecule and the immunogenic agent. Advantageously, the coupling comprises various steps which are:
to couple the immunogenic agent and the linker,
to couple the immunogenic agent, coupled to the linker, to the circulating molecule.

Preferably, the immunogenic agent coupled to the linker is present in excess during the coupling with the circulating molecule.

In one embodiment of the method of the invention, the circulating molecule and the immunogenic agent are in lyophilized form and are solubilized before being used for the preparation of the immunogenic conjugate.

In one embodiment of the method of the invention, the coupling is preferably carried out in a standard saline solution, the pH of which ranges between 7 and 8.5, in particular a PBS buffer, with stirring, for a period of between 1 h and 24 h at ambient temperature.

In one embodiment of the method of the invention, the solution comprising the immunogenic conjugate is subsequently purified on a steric exclusion column, or preferably dialyzed or ultracentrifuged, preferably at a cutoff threshold of less than 25 000 Da in the case of a Fab fragment, and at a cutoff threshold of less than 75 000 Da in the case of a whole antibody, and then optionally lyophilized.

A subject of the invention is also the use of an immunogenic conjugate for inducing and/or amplifying an immune response in a mammal, in particular a complement-dependent cytotoxicity response.

The subject of the invention is also the use of an immunogenic conjugate of the invention for the preparation of a medicament for use in treating cancers, in particular myeloma, breast cancer or lymphomas, or autoimmune diseases by targeting an autoreactive lymphocyte.

The invention will be understood more clearly from the examples which follow. These examples are given by way of illustration of the subject of the invention, of which they in no way constitute a limitation.

In the examples which follow, reference will be made to the attached figures, in which:

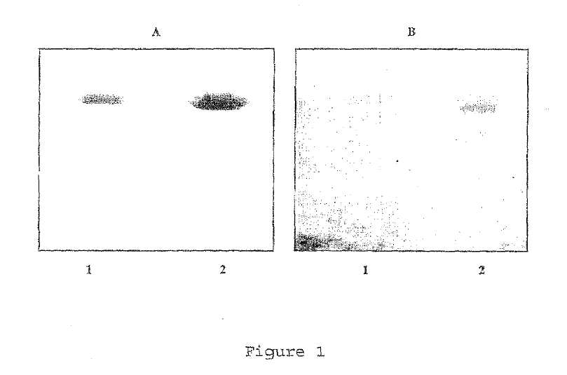

FIG. 1 shows the verification of the grafting of the Galα(1-3)Galβ(1-4)GlcNH₂ trisaccharide to Fab fragments of rituximab using sulfo-SMCC,

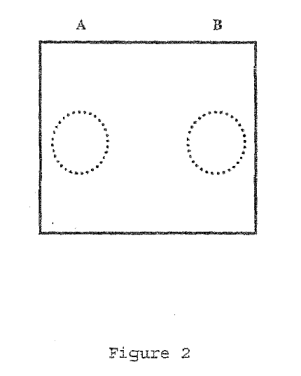

FIG. 2 shows the dot-blotting analysis of the bioconjugation of the Galα(1-3)Galβ(1-4)GlcNH₂ motif on Fab fragments of rituximab using the lectin MOA,

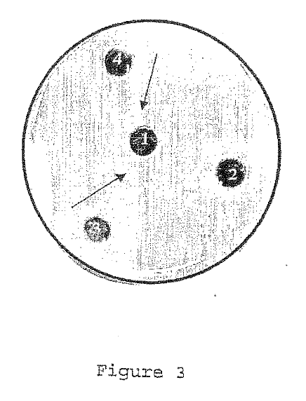

FIG. 3 shows the immunoprecipitation of Fab fragments of rituximab or of Fab fragments coupled to the α-Gal epitope on a 1% agarose gel,

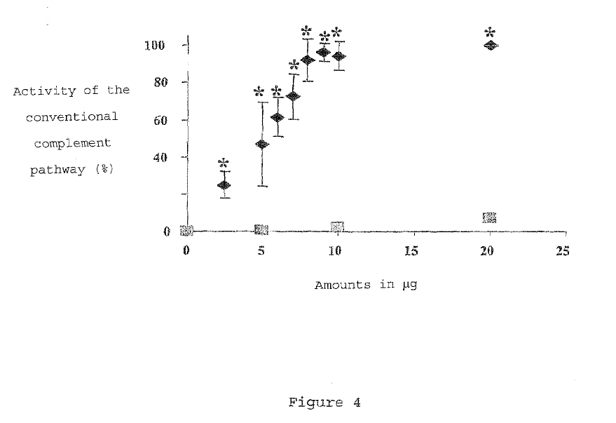

FIG. 4 shows the evaluation of the activation of the complement system with the Fab fragments and the Fab fragments coupled to the xenoantigen Galα(1-3)Galβ(1-4)GlcNH₂,

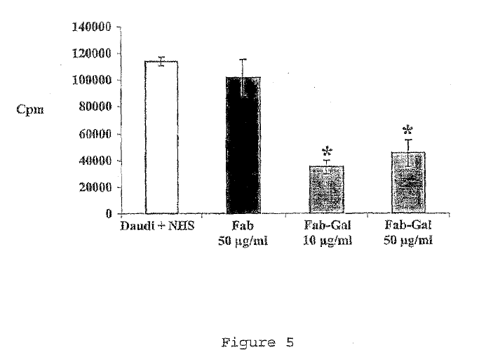

FIG. 5 shows the evaluation of the cell proliferation of the Daudi tumor line by incorporation of tritiated thymidine in the presence of 50 μg/ml of Fab fragments or of 10 μg/ml and of 50 μg/ml of Fab fragments coupled to the α-Gal epitope,

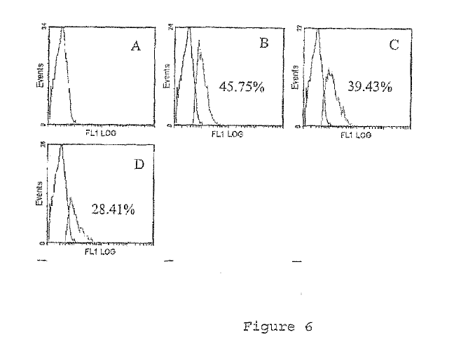

FIG. 6 shows the flow cytometry analysis of the recognition of CD20 at the surface of Daudi cells with Fab fragments of rituximab, and its conjugates with the A blood group and the B blood group,

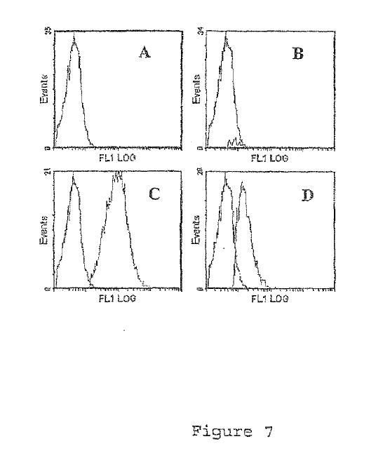

FIG. 7 shows the flow cytometry analysis of the recognition of the A blood group (D) and B blood group (C) present on the Fab fragments of rituximab by the Bandeiraea simplicifolia lectin.

### EXAMPLE 1

### Preparation of a Fab-Gal Immunogenic Conjugate

### 1-Fab and Fc Antibody Fragmentation

20 mg of rituximab are digested with 0.5 ml of immobilized papain (Pierce) in a phosphate Na₂ EDTA buffer (10 mM, pH 7) in the presence of a catalyst such as cysteine (10 mM). The papain used is immobilized on agarose beads in order to facilitate its removal at the end of the enzymatic reaction. Enzymatic kinetics made it possible to determine an optimal digestion time of 24 hours. The enzymatic reaction is stopped by the addition of 3 ml of Tris-HCl buffer (10 mM, pH 7.5). The papain immobilized on agarose beads is separated from the various fragments by centrifugation for 10 minutes at 5000 g. These fragments are subsequently purified on a protein A column and then concentrated by means of a centricon, the cutoff threshold of which is 5000 Da. The antibody fragments derived from the enzymatic digestion are visualized by electrophoresis: sodium dodecyl sulfate-polyacrylamide gel electrophoresis (SDS-PAGE) 10% under denaturing conditions.

### 2-Modification of a Gal-α(1,3)-Gal-β(1,4)-GlcNAc Trisaccharide

The trisaccharide is deacetylated in order to reveal amine groups (NH₂) onto which linker arms will be grafted.

The trisaccharide, preferably in lyophilized form, is solubilized in a solution of sodium hydroxide (1M) containing 1% of sodium tetraborohydride (NaBH₄). After 1 hour at 80° C., a solution of glacial acetic acid and then water are added to the reaction mixture in order to neutralize the NaBH₄. The deacetylated trisaccharide is subsequently purified by steric exclusion on a P2 column (Biorad) with milliQ water as eluent.

### 3-Analysis of the Formation of Amine Groups on the Deacetylated Galα(1-3)Galβ(1-4)GlcNAc by Thin Layer Chromatography

Thin layer chromatography (TLC) is based mainly on adsorption phenomena: the mobile phase is a solvent or a mixture of solvents, which progresses along a stationary phase consisting of a silica gel fixed on a glass plate or on a semi-rigid sheet of plastic or of aluminum. The amine functions are revealed with ninhydrin.

The natural trisaccharide (Galα(1-3)Galα(1-4)GlcNAc) and the deacetylated trisaccharide (Galα(1-3)Galα(1-4)GlcNH₂) are deposited onto the silica plate (Merck, France). The elution is carried out with a butanol/water/ethanol mixture (1/1/1, v/v/v). The plate is dried in an oven at 37° C. and the amine functions are then revealed with a solution of ninhydrin at 0.2% (w/v) in ethanol, and the monosaccharides are revealed with an H₂SO₄/ethanol/water mixture (v/v/v).

### 4-Preparation of the Fab Fragments for Grafting

Free thiol functions (SH) are introduced onto primary amines of the Fab fragments. Commercially available reagents such as SATA (N-succinimidyl-S-acetylthio-acetate) or Traut's reagent, may be used. Preferably, two free thiol functions per Fab molecule are introduced.

These modified Fab fragments are subsequently ultrafiltered (cutoff threshold 5000 Da) against a PBS buffer, pH 7.2, containing 5 mM EDTA. The free SH functions are assayed using Ellman's reagent (Pierce).

5-Coupling of the Deacetylated Trisaccharide onto the Modified Fab Fragments

The coupling of the trisaccharide and of the Fab fragments is carried out in the presence of a linker: sulfo-SMCC comprising a maleimide function which reacts specifically with the free thiol functions of the functionalized Fabs and an N-hydroxysuccinimide ester function (NHS) which reacts specifically with the primary amines of the deacetylated trisaccharide.

In order to prevent cross reactions between the primary amine functions of the trisaccharide and those of the Fab fragments, the coupling reaction is carried out in the following way:
a solution of deacetylated trisaccharide is reacted with the sulfo-SMCC in a PBS buffer, pH 8.5, for a period of 2 h, at ambient temperature,
this grafted motif can be separated from the deacetylated trisaccharide and from the nongrafted linker on a P2 steric exclusion column,
the reaction mixture of the preceding step is reacted with the Fab fragments for 2 h at ambient temperature in PBS, pH 7.2, the trisaccharide-sulfo-SMCC being in excess compared with the Fab fragments,
once coupled, the Fab-Gal immunogenic conjugate is ultrafiltered against PBS buffer, pH 7.2 (cutoff threshold 10 000 Da).

The concentration of Fab-Gal immunogenic conjugate is determined by absorbance at 280 nm and by the Bradford method (Biorad kit).

6-Characterization of the Grafting by Staining of the Galα.(1-3)Galβα. (1-4)GlcNH₂ Trisaccharide with Schiff's Reagent

After migration of the Fab and Fab-Gal fragments by SDS-PAGE, the gels are stained by two different techniques, one for detecting proteins, the other for detecting oxidized oligosaccharides. In fact, periodate oxidation of terminal monosaccharides makes it possible to create aldehyde functions which will be able to be labeled with Schiff's reagent and give a pink coloration (Dubray and Bezard, 1982). FIG. 1 shows the verification of the grafting of the Galα(1-3)Galβ(1-4)GlcNH₂ trisaccharide to the Fab fragments of rituximab using sulfo-SMCC, by the Coomassie blue (A) and Schiff's reagent (B) staining techniques. Lanes 1 correspond to the Fab fragments of natural rituximab, and lanes 2 correspond to the rituximab Fab fragments coupled to the Galα(1-3)Galβ(1-4)GlcNH₂ trisaccharide.

### SDS-PAGE Electrophoresis

The electrophoresis is carried out in a 14% (w/v) acrylamide gel under denaturing conditions with neither heating nor the addition of β-mercaptoethanol according to the method described by Laemmli (1970) using the Mini protean 3 system (Biorad, France).

Staining of Proteins with Coomassie Blue

The proteins in the gel are revealed by staining with Coomassie blue according to a standard protocol (FIG. 1A): the gel is, firstly, immersed in a staining buffer for 30 min with agitation and then destained through several baths in a destaining buffer until the appearance of clear bands.

Staining of Monosaccharides with Schiff's Reagent

Staining with Schiff's reagent (FIG. 1B) makes it possible to reveal the oligosaccharide structures: the gel is first of all fixed for 1 to 2 h in an acetic acid/methanol/mQ water mixture (10/35/55 (v/v/v)). It is then immersed for 1 h in the dark in the oxidation buffer (0.7 g of periodic acid in 100 ml of 5% (v/v) acetic acid) in order to create aldehyde functions. After rinsing with water, the gel is incubated in a solution of Schiff's reagent (Sigma-Aldrich, France), which reacts with the aldehyde functions thus formed, for 1 h at 4° C. Finally, the gel is destained through several baths of 7.5% (v/v) acetic acid at ambient temperature.

7-Characterization of the α-Gal Antigenic Motif Grafted onto the Fab Fragments, by Dot-Blotting

The MOA (Marasmium oreades agglutinin) lectin is a protein which specifically recognizes the Galα(1-3)Gal motif (Winter et al., 2002, The mushroom Marasmius oreades lectin is a blood group type B agglutinin that recognizes the Galα1,3Gal and Galα1,3Galβ1,4GlcNAc porcine xenotransplantation epitopes with high affinity, J. Biol. Chem. 277(17): 14996-5001). Consequently, the xenoantigen Galα(1-3)Galα(1-4)GlcNH₂ coupled to the Fab fragments of rituximab blotted onto a nitrocellulose membrane can be specifically labeled with this lectin. Since the latter is coupled to HRP (horseradish peroxidase), it is possible to reveal this complex by amplified chemiluminescence (ECL) which allows the enzymatic formation of an acridinium ester, which is degraded to an excited compound that releases its energy in the form of light at a maximum wavelength of 430 nm and that can be visualized on a photosensitive film.

10 μg of the various antibody fragments are blotted onto a 0.22 μm nitrocellulose membrane (Biorad, France) using the Bio-Dot apparatus (Biorad, France). The nonspecific sites are subsequently blocked by incubation of the membrane in a TBST buffer (20 mM Tris-HCl containing 500 mM of NaCl and 0.05% of Tween 20, pH 7.5) containing 5% of BSA (w/v), for 1 h at ambient temperature. The membrane is subsequently washed with 3 baths of TBST each for 5 min, and then incubated with the MOA lectin coupled to HRP (EY Laboratories, Biovalley, France) ( 1/100 000^{th} in TBST containing 5% of BSA (w/v)) for 45 min at ambient temperature. The membrane is again washed with 3 baths of TBST, each for 5 min. Finally, the revelation is carried out by chemiluminescence (ECL₊ revealing kit, Amersham, France) and then development on a photosensitive film (Hyperfilm ECL, Amersham, France). FIG. 2 shows the dot-blotting analysis of the bioconjugation of the Galα(1-3)Galβ(1-4)GlcNH₂ motif on the Fab fragments of rituximab using the MOA lectin. A corresponds to the Fab fragments of natural rituximab, and B corresponds to the rituximab fragments coupled to the Galα(1-3)Galβ(1-4)GlcNH₂ motif.

### 8-Immunoprecipitation on Agarose Gel: Ouchterlony Method

This technique is based on the fact that proteins, and in particular immunoglobulins, are capable of migrating more or less rapidly in an agarose gel (1% in PBS, pH 7.2). The immunoglobulins immunoprecipitate when they recognize the antigen. The immunocomplex formed by an immunoglobulin and its antigen is visualized by a precipitation arc. Using this method, it is possible to verify that the modified or unmodified trisaccharides are effectively recognized by immunoglobulins of human serum (normal human serum diluted to 1/50^{th} in PBS and approximately 50 μg of Fab-Gal versus Fab in each well). 100 μl of normal human serum ( 1/20^{th}) are deposited in the central well of the agarose gel. 70 μg of the various samples are deposited in the other wells, approximately 1 cm from the central well, and the whole is incubated for 24 to 48 hours under a humid atmosphere and at ambient temperature. FIG. 3 shows the result of this immunoprecipitation. Well 1 corresponds to the normal human serum, well 2 to the Fab fragments of natural rituximab, and wells 3 and 4 to the rituximab Fab fragments coupled to the α-Gal epitope.

### EXAMPLE 2

### Evaluation of the Activity of a Fab-Gal Immunogenic Conjugate on the Hemolytic Capacity of a Normal Human Serum at the CH₅₀

The activation of the complement system in a normal human serum (NHS) is triggered by the introduction of sheep red blood cells (EA) pre-coated with rabbit antibodies. In the presence of human serum, the rabbit antibodies recognize the sheep red blood cells as foreign and trigger the activation of the conventional complement pathway. This activation leads to the formation of conventional C3 convertase. The activation of this pathway results in the formation of the final lytic complex creating pores in the cell membrane of the target cell at the surface of the red blood cells. This complex allows the red blood cells to rupture and the hemoglobin to be released. The activation of the complement system is measured in this experimental system by assaying the amount of hemoglobin released, by absorbance at a wavelength of 414 nm. In this assay, the activator which corresponds to the sheep red blood cells also serves as agent revealing the activation by virtue of the hemoglobin released.

The overall strategy for determining the activity of a molecule on the complement system is the following: firstly, we will determine the activity of the molecules on the hemolytic capacity of a normal human serum (NHS) at the CH₅₀ (concentration of NHS for which a maximum of 50% of red blood cell lysis is obtained), making it possible to evaluate an inhibitory activity on the conventional complement system pathway. Secondly, tests on sera deficient in certain proteins of the complement system will make it possible to evaluate the ability of the various molecules to activate the complement system. All these measurements are carried out at the end point, after the entire enzymatic cascade has occurred.

The action of molecules on EAs at the CH₅₀ makes it possible to observe any possible inhibition of the conventional complement system pathway. In fact, the NHS incubated with the molecule studied contains all the proteins of the complement cascade. Thus, if the EAs are lyzed, this means either that the molecule activates the complement system or that it has no action. On the other hand, if the cells are not lyzed, this indicates that there has been inhibition of the conventional complement system pathway by the molecule. It will therefore be possible to calculate a percentage inhibition as a function of the concentration of this molecule.

If the molecule is not inhibitory, it is necessary to determine whether it is an activating molecule or whether it has no action on the complement system. In order to determine this, the molecule is then incubated firstly with NHS. If it activates the complement system, the proteins will be “consumed” during this incubation. If not, the proteins will still be present in the mixture. Next, the whole is again incubated with a serum deficient in or depleted of one of the proteins of the complement cascade, and EAs. If the molecule has no action on the complement system, the NHS proteins not consumed will restore the hemolytic capacity of the deficient serum and the EAs will be lyzed. Conversely, if the proteins were consumed during the activation of the complement system, the hemolytic capacity of the deficient serum will not be restored and the red blood cells will not therefore by lyzed. It will thus be possible to determine a percentage activation of the conventional complement pathway as a function of the concentration of the molecule studied.

By means of these various assays, it is possible to determine a potential inhibitory or activating activity of molecules on the complement system.

### 1-Determination of the CH₅₀

350 μl of NHS (Etablissement Français du Sang (EFS) [French Blood Bank], France) at various concentrations in VBS²⁺are incubated with 450 μl of VBS²⁺and 200 μl of EA diluted to 1/20^{th} for 45 min at 37° C. The controls, corresponding to 0% (L₀) and 100% (L₁₀₀) lysis, are incubated under the same conditions. They are prepared by incubating 800 μl of VBS²⁺ in the presence of 200 μl of EA diluted to 1/20^{th}.

2 ml of cold 0.15M NaCl are added to each tube except that corresponding to L₁₀₀ (to which 2 ml of mQ water are added in order to have total lysis). After centrifugation for 15 min at 600 g, the OD of the supernatant is measured at 414 nm.

### 2-Evaluation of the Activity of Molecules on the Hemolytic Potency of a Normal Human Serum at the CH₅₀

This experiment makes it possible to evaluate the ability of the molecule tested to inhibit the conventional complement system pathway.

350 μl of NHS at the CH₅₀ are incubated with 450 μl of VBS²⁺containing 0 to 100 μg of the test molecule and 200 μl of EA (diluted to 1/20^{th}) for 45 min at 37° C. in a waterbath. The controls, corresponding to 0% (L₀) and 100% (L₁₀₀) lysis, are incubated under the same conditions. They are prepared by incubating 800 μl of VBS²⁺ in the presence of 200 μl of EA diluted to 1/20^{th}.

After the addition of 2 ml of cold NaCl at 0.15M (except for that corresponding to L₁₀₀, to which 2 ml of mQ water are added) and centrifugation for 15 min at 600 g, the OD of the supernatants is measured at 414 nm.

The hemolytic capacity of the serum, called Y, is then determined in the presence or absence of the molecules tested:

Y=(ODₛₐₘₚₗₑ−OD_{L0})/(OD_{L100}−OD_{L0})

The loss of hemolytic capacity of the samples containing the various molecules tested directly accounts for the inhibition of complement:

DY=Y_{0 mg of molecule tested}−Y_{x mg of molecule tested}

% inhibition=(DY/Y_{0 mg of molecule tested})×100

The CH₅₀ is the concentration of NHS to have 50% lysis of sensitized sheep red blood cells. In the various experiments carried out during this study, the CH₅₀ is obtained with a 1/100^{th} dilution of NHS.

A first experiment allows us to evaluate the hemolytic capacity of the NHS at the CH₅₀ when it is incubated with 100 μg of Fab fragments (used as controls) or of Fab-Gal fragments. We intentionally chose a relatively high concentration of Fab or Fab-Gal fragments in order to see any activity of these molecules on the hemolytic potency of the NHS.

The results obtained, represented in the figure hereinafter, indicate that the NHS incubated with the Fab fragments or the Fab-Gal fragments maintains a hemolytic potency of 50%. These molecules consequently have no inhibitory activity on the conventional complement system pathway, but this experiment does not allow us to estimate a potential activation.

CH50 without Preincubation

| | Serum alone | | |
|---|---|---|---|
| Experiments | (control) | Fab | Fab Gal |
| 1 | 17 | 17 | 20 |
| 2 | 17 | 18 | 25 |
| 3 | 30 | 29 | 50 |

A second experiment is then carried out by preincubating 100 μg of Fab or Fab-Gal fragments with NHS diluted to 1/100^{th}, for 45 min at 37° C., and then evaluating the hemolytic capacity of this serum. We observed no modification of the hemolytic capacity of the serum preincubated with the Fab fragments alone, which remains 50% (figure “CH₅₀ with preincubation”). Once again, said Fab fragments have no activity on the conventional complement system pathway. However, when the NHS is preincubated with the soluble Fab-Gal fragments, there is a significant reduction in hemolytic potency.

CH50 with Preincubation

| | Serum alone | | |
|---|---|---|---|
| Experiments | (control) | Fab | Fab Gal |
| 1 | 33 | 16 | 5 |
| 2 | 24 | 5 | 1 |
| 3 | 86 | 83 | 73 |
| 4 | 95 | 53 | 3 |
| 5 | 96 | 54 | 2 |

This result can be explained by the fact that, during the preincubation, there is recognition of the α-Gal xenoantigen by the natural Igs, and more particularly IgMs, present in the NHS. There is then formation of immunocomplexes due to the divalence of the epitopes grafted to the Fab generating a high capacity to activate the conventional complement system pathway. This activation leads to a “consumption” of all the proteins of the cascade, and the NHS, since it then lacks these proteins during the incubation with the sensitized sheep red blood cells, can no longer therefore have hemolytic activity.

Thus, by determining the activity of these various molecules on the hemolytic activity of a normal human serum at the CH₅₀, by carrying out or not carrying out a preincubation of the Fab-Gals with the NHS, we demonstrate that these bioconjugates have no inhibitory activity on the conventional complement system pathway. We also have strong reasons to think that they might have an ability to activate this system. In order to confirm this hypothesis, we will study the activation of the complement system by means of other hemolytic assays using a guinea pig serum deficient the complement C4 protein.

### EXAMPLE 3

### Determination of the Ability of a Molecule To Activate the Conventional Complement Pathway

In order to supplement the demonstration that these novel Fab-Gal immunogenic conjugates have an ability to activate the conventional complement pathway, EA lysis assays in serum deficient in one of the proteins of the complement cascade were carried out. These assays make it possible to determine the percentage activation of the conventional complement pathway as a function of the concentration of various oligosaccharides. This makes it possible to measure the restoration of the hemolytic capacity of a serum deficient in an identified protein (C4, C2 or C1q) and thus to calculate the percentage activation of the conventional complement system pathway as a function of the concentration in the molecule.

15 μl of NHS (diluted to 1/20^{th}) are incubated with various amounts of the test molecule for 45 min at 37° C. A negative control (control 0 mg), which makes it possible to measure the cell lysis only by the NHS, is carried out without molecule, under the same experimental conditions. The whole is then incubated with 100 μl of serum deficient in a known protein of the complement cascade (the dilution of which was predetermined so as to have 90% cell lysis under these experimental conditions and without molecule), 100 μl of EA (diluted to 1/20^{th}) and VBS²⁺ so as to have a final volume of 500 μl, for 45 min at 37° C. in a waterbath.

The controls, corresponding to 0% (L₀) and 100% (L₁₀₀) lysis, are incubated under the same conditions. They are prepared by incubating 400 μl of VBS²⁺ in the presence of 100 μl of EA diluted to 1/20^{th}.

After the addition of 2 ml of cold NaCl at 0.15M (except for that corresponding to L100, to which 2 ml of mQ water are added) and centrifugation for 15 min at 600 g, the OD of the supernatants is measured at 414 nm.

Aggregated IgGs, treated under the same experimental conditions, serve as positive controls during this experiment and make it possible to validate the experimental model. They are in fact very effective activators of the conventional complement pathway.

The ability to activate the conventional complement pathway is then determined according to the following calculations:

% lysis=(ODₛₐₘₚₗₑ−OD_{L0})/(OD_{control 0 mg})×100

% activation=100−% lysis

This experiment allows us to evaluate the ability of various molecules to activate the conventional complement system pathway. It is based on the ability of NHS incubated with various concentrations of Fab or Fab-Gal to restore the hemolytic potency of a guinea pig serum deficient in one of the complement proteins, the latter being in this case the C4 protein.

The results, shown in the figure, indicate that the Fab-Gal fragments activate the complement system, whereas the Fab fragments have no activity in this system. In fact, we obtain 100% activation of the conventional complement pathway with the Fab-Gal fragments, with an amount of 20 μg, whereas the Fab fragments do not possess any ability to activate this system.

We therefore demonstrate very clearly that the Galαl-3Gal motif, grafted and correctly presented on the Fab fragments of rituximab, is recognized by serum Igs which activate the complement system. These results allow us to confirm the hypothesis that the serum Igs are predominantly natural IgMs specific for the α-Gal epitope, having a very high capacity to activate the conventional complement system pathway.

These experiments can, however, be supplemented with other hemolytic assays using sera deficient in IgM in order to confirm that the activation indeed comes from immunoglobulins of this type.

Result: Activation of the Conventional Complement System Pathway by Fab Fragments Alone and the FabGal Immunogenic Conjugate in Accordance with the Invention

This assay reveals that the percentage activation of complement is much higher when the Fab fragments are coupled to this oligosaccharide motif.

Result: Activation of Complement as a Function of the Concentration of Fab-Gal Immunogenic Conjugate in Accordance with the Invention

| | % | % | | |
|---|---|---|---|---|
| Amount | activation | activation | Mean over | |
| FabGal | assay 1 | assay 2 | 2 assays | SD |
| 0 | 0 | 0 | 0 | 0 |
| 2.5 | 30 | 20 | 25 | 7.0 |
| 5 | 63 | 31 | 47 | 22.6 |
| 6 | 69 | 54 | 61.5 | 10.6 |
| 7 | 81 | 64 | 72.5 | 12.0 |
| 8 | 100 | 84 | 92 | 11.3 |
| 9 | 100 | 93 | 96.5 | 4.9 |
| 10 | 100 | 89 | 94.5 | 7.7 |
| 20 | 100 | 100 | 100 | 0 |

As demonstrated by the table above, the oligosaccharide motif coupled to the Fab fragments allows the conventional complement system pathway to be activated.

FIG. 4 shows an illustration of the two results given above: the Fab fragments of natural rituximab are represented by squares and the rituximab fragments coupled to the Galα(1-3)Galβ(1-4)GlcNH₂ xenoantigen are represented by diamonds.

### EXAMPLE 4

### Evaluation of the Biological Activity of a Fab-Gal Immunogenic Conjugate: Cell Proliferation Inhibition Assays

This assay makes it possible to determine the ability of the Fab-Gal immunogenic conjugates to inhibit the proliferation of a tumor cell line. The cell models used in this study are the Daudi (E. Klein and G. Klein, 1967) and Raji (R. J. V. Pulvertaft, 1963) cancer lines. The cells are cultured in 50 cm³ or 275 cm³ flasks at a concentration ranging from 3×10⁵ to 1×10⁶ cells/ml. The Daudi line is cultured in an RPMI medium supplemented with 1% glutamine, 1% antibiotics and 20% fetal calf serum (FCS, Gibco) heat-decomplemented at 56° C. for 30 min.

The technique used is a technique for measuring the incorporation of tritiated thymidine into DNA of dividing cells. The cells are therefore incubated with Fab fragments (10 μg/ml) (control) and two concentrations (10 and 50 μg/ml) of Fab-Gal immunogenic conjugates, for 24 h, in a medium containing 20% of NHS and radioactive thymidine (thymidine being a base of the DNA) (1 μl of thymidine per well). The cells which proliferate normally will incorporate radioactive thymidine that can be assayed by means of a scintillation counter. The cells whose cell proliferation is affected will incorporate less radioactive thymidine.

FIG. 5 shows the evaluation of the cell proliferation of the Daudi tumor line by incorporation of tritiated thymidine in the presence of 50 μg/ml of Fab fragments or 10 μg/ml and 50 μg/ml of Fab fragments coupled to the α-Gal epitope.

The Fab fragments alone do not make it possible to significantly inhibit the cell proliferation, whereas the Fab-Gal immunogenic conjugates of the invention make it possible to inhibit cell proliferation.

### EXAMPLE 5

### Preparation of a Fab-Blood Group Immunogenic Conjugate

### 1-Coupling of the Trisaccharides of the A and B Blood Groups to the Modified Fab Fragments

The coupling of the group A (or group B) trisaccharide and of the Fab fragments is carried out in the presence of the sulfo-SMCC linker comprising a maleimide function which reacts specifically with the free thiol functions of the Fabs and an N-hydroxysuccinimide (NHS) ester function which reacts specifically with the primary amines of the trisaccharide.

In order to prevent cross reactions, the coupling reaction is carried out in the following way:
a solution of trisaccharide (1 eq.) is reacted with the sulfo-SMCC (1.01 eq.) in a PBS buffer, pH 8, for a period of 2 h, at ambient temperature,
the reaction mixture of the previous step is reacted with the Fab fragments for 1 h at ambient temperature in PBS, pH 7.2, the trisaccharide-sulfo-SMCC being in excess compared with said Fab fragments. The coupling reaction is monitored by UV at 330 nm in order to observe the consumption of the maleimide functions.

Once coupled, the Fab-group A (or Fab-group B) immunogenic conjugate is ultrafiltered against PBS buffer, pH 7.2 (cutoff threshold of 10 000 Da). The concentration of Fab-group A (or Fab-group B) immunogenic conjugate is determined by the Bradford method (Biorad kit).

The bioconjugate is characterized according to the same techniques as above.

### 2-Western Blotting of Fab-Group B

The proteins are identified according to their molecular weights by SDS-PAGE electrophoresis. For the Western blotting, once the electrophoresis has been carried out, the proteins are separated but remain in the polyacrylamide gel. The various proteins contained in the gel are then adsorbed onto a nitrocellulose membrane; for this, the electrodes sandwich the gel and the nitrocellulose membrane surrounded by filters. After the transfer, the membrane is stained with Ponceau red, a protein-specific dye, in order to verify that the electrophoresis and the transfer have taken place correctly. Before using the MOA lectin-HRP as group-B-specific probe, all the potential binding sites not used on the membrane must be blocked using Tween-20 and BSA, which minimizes nonspecific protein-protein and protein-matrix adsorption. The nitrocellulose membrane containing the proteins is then brought into contact with the MOA lectin-HRP for 45 minutes and then, after several washes, the position of the MOA lectin/group B trisaccharide complex is detected by chemiluminescence.

### EXAMPLE 6

### Flow Cytometry Analysis of the Fab-Blood Group Immunogenic Conjugate Recognition for CD20

1-Use of a Secondary Antibody which Specifically Recognizes Fragments of the Murine Kappa Chain of Rituximab Fab Fragments

The recognition, by the Fab fragments of rituximab and conjugates thereof with blood groups A and B, of the CD20 protein present at the surface of Daudi cancer cells obtained from B lymphomas (line originating from the ATCC, American Type Culture Cells) was examined by flow cytometry.

10⁵ cells in suspension in 100 μl of PBS/1% BSA were incubated in the presence of PBS (negative control) or of 10 μg of Fab, of Fab-group A or of Fab-group B, for 20 min at ambient temperature (22° C.). After two washes in PBS/1% BSA in order to remove the excess antibody fragment, 100 μl of FITC-conjugated goat anti-mouse kappa antibody (ICL), diluted to 1/10^{th} in PBS buffer/1% BSA, were added for 20 minutes at ambient temperature. Two washes in PBS buffer/1% BSA were then carried out and the analysis of the cells, taken up in 500 μl of PBS buffer, was performed on approximately 5000 cells by flow cytometry (Becton Dickinson equipped with an argon laser with a wavelength of 488 nm for analysis of FITC). FIG. 6 shows the flow cytometry analysis of the recognition of CD20 at the surface of Daudi cells by Fab fragments of rituximab (B), and conjugates thereof with blood group A (C) and blood group B (D). The percentage of fluorescent cells (positive with labeling by the secondary antibody) is indicated in the legend.

When the recognition obtained with the natural Fab fragments and the Fab-group A and Fab-group B conjugates is compared, a slight decrease in the recognition by the Fab-group A and Fab-group B conjugates is observed, which may be due either to a less effective recognition, by the conjugates, for their target, CD20, or to a less effective recognition, by the secondary antibody, of the kappa chain of murine origin present on the Fab parts of the conjugates. This can be explained by steric hindrance induced by the presence of the blood groups on the Fab.

### 2-Use of a Lectin Specific for Blood Group B

The recognition, by the Fab fragments of rituximab and conjugates thereof with blood groups A and B, of the CD20 protein present at the surface of Daudi cancer cells obtained from B lymphomas (line originating from the ATCC, American Type Culture Cells) was examined by flow cytometry.

10⁵ cells in suspension in 100 μl of PBS, 0.5 mM CaCl₂ were incubated in the presence of PBS (negative control) or of 10 μg of Fab, of Fab-group A or of Fab-group B. for 20 min at ambient temperature (22° C.). After two washes in PBS, 0.5 mM CaCl₂ in order to remove the excess antibody fragments, 100 μl of Bandeiraea simplicifolia lectin (Sigma) conjugated to FITC, at 10 mg/l in PBS buffer, 0.5 mM CaCl₂, were added for 20 minutes at ambient temperature. Two washes in PBS buffer, 0.5 mM of CaCl₂ were then carried out and the analysis of the cells, taken up in 500 μl of PBS buffer, was performed on approximately 5000 cells by flow cytometry (Becton Dickinson equipped with an argon laser with a wavelength of 488 nm for the analysis of FITC). FIG. 7 shows the flow cytometry analysis of the recognition of blood groups A (D) and B (C) present on the rituximab Fab fragments by the Bandeiraea simplicifolia lectin. Represented in A and B are the histograms obtained with the Daudi cells alone and in the presence of rituximab Fab fragments, respectively.

The percentage of fluorescent cells (positive with labeling by the lectin) is 94.9% for the Fab-blood group B conjugate and 66.7% for the Fab-blood group A conjugate.

The lectin used has a high affinity for terminal α-D-galactosyl residues (present on blood group B) and a lower affinity for terminal N-acetyl-α-D-galactosaminyl residues (present on blood group A). Thus, the differences in percentage of fluorescent cells obtained may be explained by the reasons indicated above.

By means of this assay, we demonstrate not only that the Fab-blood group conjugates contain motifs that are correctly exposed since they can be detected by the lectin, but also that the CD20 antigen is recognized by these same conjugates.

1. An immunogenic conjugate comprising a target cell-specific circulating molecule and at least one immunogenic agent, said immunogenic agent being coupled by any appropriate means to the circulating molecule.

2. The immunogenic conjugate as claimed in claim 1, characterized in that said immunogenic agent is coupled to the circulating molecule by a covalent bond.

3. The immunogenic conjugate as claimed in claim 1, characterized in that said immunogenic agent is coupled to the circulating molecule by means of a linker, preferably a heterobifunctional linker.

4. The immunogenic conjugate as claimed in claim 3, characterized in that said linker is chosen from sulfo-SMCC.

5. The immunogenic conjugate as claimed in claim 1, characterized in that said circulating molecule is an antibody, a monospecific monoclonal antibody or a multispecific monoclonal antibody.

6. The immunogenic conjugate as claimed in claim 1, characterized in that said circulating molecule is an antibody fragment, in particular a Fab fragment.

7. The immunogenic conjugate as claimed in claim 1, characterized in that said immunogenic agent comprises at least one oligosaccharide comprising at least one motif capable of being recognized by at least one antibody and/or a soluble protein of human complement and/or by a receptor expressed at the surface of immune system effector cells.

8. The immunogenic conjugate as claimed in claim 1, characterized in that said immunogenic agent is an oligosaccharide comprising the GalαGal epitope.

9. The immunogenic conjugate as claimed in claim 1, characterized in that said immunogenic agent is a disaccharide, in particular Gal-(α1,3)-Gal.

10. The immunogenic conjugate as claimed in claim 1, characterized in that said immunogenic agent is a trisaccharide, in particular Gal-(α1,3)-Gal-R where R is a monosaccharide, an amine function, a hydroxyl function, a carboxylate function, or an organic or inorganic substituent.

11. The immunogenic conjugate as claimed in claim 1, characterized in that said immunogenic agent is a blood group oside structure.

12. The immunogenic conjugate as claimed in claim 1, characterized in that said antibody or antibody fragment is rituximab or a rituximab fragment.

13. A method for preparing the immunogenic conjugate as described in claim 1, comprising:
optionally, the creation of reactive functions in the circulating molecule and in the immunogenic agent,
a coupling reaction between the circulating molecule and at least one immunogenic agent.

14. The method for preparing the immunogenic conjugate as claimed in claim 13, characterized in that the circulating molecule is modified with SATA (N-succinimidyl-S-acetylthioacetate) or 2-iminothiolane (Traut's reagent).

15. The method for preparing the immunogenic conjugate as claimed in claim 13, characterized in that the immunogenic agent is modified by the addition of a solution of sodium hydroxide and sodium borohydride.

16. The method for preparing the immunogenic conjugate as claimed in claim 13, characterized in that the coupling is carried out in the presence of a linker, preferably a heterobifunctional linker, very preferably sulfo-SMCC.

17. The method for preparing the immunogenic conjugate as claimed in claim 13, characterized in that the coupling of the circulating molecule and of the immunogenic agent is carried out in various steps, which are:
to couple the immunogenic agent and the linker,
to couple the immunogenic agent coupled to the linker, preferably present in excess, to the circulating molecule.

18. A method for amplifying an immune response in a mammal, in particular a complement-dependent cytotoxicity response, comprising administering the immunogenic conjugate of claim 1.

19. A method for treating cancers, in particular myeloma, breast cancer or lymphomas, or autoimmune diseases, comprising administering the immunogenic conjugate of claim 1.

20. The immunogenic conjugate as claimed in claim 2, characterized in that said circulating molecule is an antibody, a monospecific monoclonal antibody or a multispecific monoclonal antibody.
